(19) United States
(12) Patent Application Publication
(10) Pub. No.: US 2008/0233207 A1
(43) Pub. Date: Sep. 25, 2008

(54) Injectable and Infusable Mercury Compositions and Methods for Treating Cancer

(76) Inventor: Yelena G. Sheptovitsky, Weston, CT (US)

Correspondence Address: GORDON & JACOBSON, P.C., 60 LONG RIDGE ROAD, SUITE 407, STAMFORD, CT, 06902, US

(21) Appl. No.: 12/132,052

(22) Filed: Jun. 3, 2008

Related U.S. Application Data
(63) Continuation-in-part of application No. 11/324,850, filed on Jan. 4, 2006.

(51) Int. Cl. A61K 33/28; A61P 35/00

(52) U.S. Cl. 424/645; 424/644

(57) ABSTRACT

An aqueous solution suitable for injection into a mammal in need thereof includes an inorganic mercury (II) containing compound present in an amount between 0.1 nanomole and 50 millimoles Hg²⁺ where the solution is sufficiently stable such that there is no precipitation from the solution for at least thirty days of a visable precipitate containing at least some of said mercury. In one embodiment, a buffer which stabilizes the pH of the solution at a desired pH between 3.0 and 7.0 is provided. In another embodiment, at least one salt is provided in amounts which provide sufficient ionic strength to the solution and coordination ligand to prevent precipitation of the mercury from the solution.

### CROSS-REFERENCE TO RELATED APPLICATIONS

This application is a continuation-in-part of U.S. Ser. No. 11/324,850 which is hereby incorporated herein in its entirety by reference.

### BACKGROUND OF THE INVENTION

1. Field of the Invention

This invention relates broadly to inorganic mercy (II) compounds for use as injectable or infusible anti-cancer drugs. More particularly, this invention relates to injectable and infusible inorganic mercury (II) compounds which have a long shelf-life and are believed efficacious in treating cancer.

2. State of the Art

As set forth in the parent application hereto, mercury has been used for centuries as an essential part of many different medicines. Prior to the advent of antibiotics, mercury chloride was used to treat syphilis. In addition, mercury-containing drugs included diuretics, antibacterial agents, antiseptics, and laxatives. In spite of a long history of mercury use in medicine, due to growing concerns about mercury toxicity in environmental and occupational exposure (Kazantzis, 2002, Med Lav 93:139-47), its use in conventional medicine is currently very limited (Mercury in Drug and Biologic Products. FDA document, Aug. 5, 2003).

Mercury toxicity has been studied extensively in medicine (Langford N. et al., 1999, J Hum Hypertens. 13(10):651-6, Toxicological profile for mercury. U.S. Department of health and human services, Public Health service, Agency for Toxic Substances and Disease Registry). Mercury is a highly toxic non-essential heavy metal that has three different species with distinct pharmacokinetics and toxicity profiles: elemental mercury metal, inorganic mercury salts and organometallic mercury compounds. Inorganic mercury salts are a far greater hazard than metallic mercury. The majority of ingested inorganic mercury salt accumulates either in the liver where it is excreted in the bile, or in the kidney, where it is excreted in the urine. The symptoms and signs of inorganic mercury poisoning are mercurial stomatitis, loosening of the teeth and renal damage. Inorganic mercury salts, however, do not cross the blood brain barrier in significant amounts as opposed to organic mercury compounds where toxicity is mostly seen in the central nervous system.

There is a vast amount of literature about toxicity of inorganic mercury in vivo and in vitro. Mercury (II) salts were reported to induce apoptosis (Duncan-Achanzar et al., 1996, J. Pharmacol. Exp. Ther. 277: 1726-1732), interfering with essential metals (Weinsberg et al., 1995, Arch. Toxicol. 69: 191-6) affecting their role as cofactors for enzymes, metabolic and transcription processes, form conjugates with proteins through their thiol groups (Sundberg et al., 1999, Toxicology 137: 169-84), cause autoimmunity at subtoxic concentrations (Pollard et al., 1997, Met. Ions Biol. Syst. 34:421-40), be mutagenic, elastogenic and teratogenic.

Surprisingly, mercury was found to be non-carcinogenic in vitro (Ariza et al., 1996, Environ. Mol. Mutagen; 27(1):30-3, Ariza et al., 1995, Environ. Mol. Mutagen; 25: 3) and it does not meet requirements to be classified as a human carcinogen (Toxicological profile of Mercury, March 1999, CAS# 7439-97-6, Agency for Toxic Substances and Disease Registry; U. California 2004 Reference List). In fact, mercury has been shown to be preferentially absorbed by cancer and pre-cancer cell nuclei (Omura et al., 1996, Acupunct. Electrother Res; 21(2):133-60, Kanai et al., 1980, Shinshu Igaku Zasshi; 28(2):221-228), and there have been some disclosures regarding the use of mercury in treating cancer.

Vorobieva, U.S. Pat. No. 5,889,048, discloses and claims the use of mercury dichloride formulated at 0.05-1.5% by weight in either dry white wine with a sugar content of 3-4% or whey with a sugar content of 3-4%, or pork fat plus natural honey and alcohol for the treatment of various cancers. According to Vorobieva, the wine, whey or pork fat in these formulations are an essential ingredient that renders the claimed pharmaceutical levels of mercury non-toxic and helps deliver it to the target tissue by forming covalent complexes through the SH groups of denatured proteins. However, Vorobieva does not contemplate or suggest the systemic delivery of non-covalently bound inorganic mercury (II) in its ionized or ionizable form for the treatment of cancer as the mercury (II) is covalently bound to denatured proteins in wine or food and these formulations are obviously limited to administration by mouth.

### SUMMARY OF THE INVENTION

A novel formulation for the treatment of cancer in mammals includes inorganic mercury (II) in its ionized or ionizable form which is contained in a stable solution of desired pH and ionic strength. Mercury in its ionized form or a form that is ionizable upon reconstitution or administration is not covalently bound to any other substances including but not limited to protein, fats and carbohydrates (e.g., sugars). Relatively large amounts of ionized or ionizable inorganic mercury (II) (e.g., 100 millimoles Hg²⁺ of mercury acetate or mercury chloride) can remain stably for a relatively long period of time in a solution having a desired pH (e.g., between 3.0 and 7.0), and sufficient ionic strength. In the case of mercury acetate, the solution may contain salt in order to provide the desired ionic strength to the solution and a ligand for mercury ion (II) coordination. Also, a buffer such as sodium acetate can be provided in the solution in order to maintain the desired pH.

The ability of the formulation to remain stable is in contrast to inorganic mercury (II) which is bound to substances such as carbohydrates, proteins and fats such as taught in U.S. Pat. No. 5,889,048 to Vorobieva. Inorganic mercury (II) which is bound as taught by Vorobieva will not retain efficacy when stored, even for a relatively short period of time, as the inorganic mercury (II) quickly precipitates out of solution. Thus, the precipitates from 0.027% and 0.3% mercury chloride formulations in wine were analyzed by inductively coupled plasma mass spectroscopy (ICP-MS) and were found to contain mercury ion at 5.6% and 24%, respectively. The depletion of inorganic mercury from wine formulation is expected to result in significant reduction of its potency as a cancer therapeutic.

The application of the formulation of the invention is believed to be effective in the treatment of a broad spectrum of cancers when introduced systemically. Systemic administration of the formulation via injection and/or transfusion is preferred. However, any route of administration that provides systemic delivery of inorganic mercury (II) is acceptable and it may include oral, parenteral, pulmonary, transdermal and local administration routes. It is believed that the mercury (II) compounds containing mercury (II) in an ionized or ionizable state can alleviate, reduce, ameliorate, or eliminate cancer, and can place or maintain in a state of remission of clinical symptoms or diagnostic markers associated with cancer.

A method for inhibiting proliferation of cancer cells, treating cancer and/or inhibiting metastases in a mammal in need thereof comprises administering at least once to the mammal the stable composition comprising inorganic mercury (II) in its ionized or ionizable form which is contained in a stable solution of desired pH and ionic strength in an amount effective to inhibit proliferation of said cancer cells, treat cancer and/or inhibit metastases. In a specific and preferred embodiment, it is administered systemically by injection or transfusion. In another specific embodiment, the composition of the present invention is administered at least twice to said mammal.

In one embodiment, the inorganic mercury (II) is present in an amount of about from 0.1 nanomole to 50 millimole Hg²⁺ per dose. In the presently preferred embodiment, the dose is for a human having a weight, and said dose contains between 0.001-100 micromole Hg²⁺/kg of the human.

In one embodiment, a treatment course of inorganic mercury (II) consists of its single administration or repeated daily or every other day administration for a certain period of time. The method in a more specific embodiment comprises an initial treatment course of said composition and at least one subsequent treatment course of said composition at least 2 days after the initial treatment course. The method in yet a more specific embodiment, comprises an initial treatment course of said composition and at least two subsequent treatment courses of said composition at least 2 days after the initial treatment course.

### DETAILED DESCRIPTION OF THE PREFERRED EMBODIMENTS

The present invention encompasses pharmaceutical compositions useful for treating cancer which comprise inorganic mercury (II) in its ionized or ionizable form which is contained in a stable solution of desired pH and ionic strength. For purposes herein, the composition is deemed to be “stable” if precipitate containing at least some of the mercury is not visible to the unaided eye for at least 30 days. Preferably, no precipitate will form for at least six months. The composition is preferably compatible with injection into the body or infusion into the bloodstream. For stability purposes, the composition has a pH of between 3.0 and 7.0, and more preferably a pH of between 3.5 and 6.0. In certain embodiments, a buffer such as sodium acetate is utilized to maintain a pH of between 4 and 5.5. It is believed that the ionic strength of the solution affects precipitation of the mercury ions. Thus, in some embodiments, a salt such as sodium chloride is provided in a desired amount to provide a desired ionic strength and a chloride ion ligand for mercury ion coordination. In other embodiments which utilize mercury chloride, the salt may not be needed.

This invention also encompasses a method for treating cancer in a mammal including a human, which comprises administering to a subject in need of such therapy a therapeutically effective amount of inorganic mercury (II). The cancers may be solid tumors or disperse cancers.

For clarity of disclosure, and not by way of limitation, the detailed description of the invention is divided into subsections which follow.

The Solution

The solution of the invention contains “inorganic mercury (II)” in a solution which is of a sufficient pH, ionic strength and coordination ligands such that the mercury does not precipitate from the solution in a manner visible to the unaided eye for at least 30 days, and more preferably at least 60 days, and most preferably at least 180 days. In one embodiment the solution contains a “buffer” which maintains the pH of the solution. In another embodiment, the solution contains a “salt” which provides desired “ionic strength” and a desired “coordination ligand”.

As used herein, “inorganic mercury (II)” refers to, in a pharmaceutically acceptable form, a compound which comprises mercury (II) in an ionized or ionizable state. The invention encompasses all pharmacologically active species of inorganic mercury (II). The term “inorganic mercury (II)” encompasses any inorganic and organic compound that generates free or coordinated mercury (II) ion in a pharmaceutical formulation or upon administration. Any ionized or ionizable mercury (II) compound(s), when used alone or in combination with other compounds, that can alleviate, reduce, ameliorate, prevent, or place or maintain in a state of remission of clinical symptoms or diagnostic markers associated with cancers, can be used in the present invention. Examples of “mercury (II) compound” include but not limited to mercury (II) salts and mercury (II) oxide. Mercury (II) salts such as mercury acetate and mercury chloride are presently preferred.

As used herein, a “buffer” refers to a pharmaceutically acceptable ionic compound which resists changes in pH of a solution. Since, according to the invention, the most desired pH is between 4.0 and 5.5, sodium acetate is a preferred buffer. However, other buffers, by way of example and not limitation, such as citric acid, glycine, lactic acid, piperazine, and MES may be used. In addition, if a different pH is desired, other pharmaceutically acceptable buffers could be utilized. In addition, deionized and reagent-quality water, typically having a pH of between 5.5 and 6.5 can act as a “buffer” if the amount of inorganic mercury added to the solution is very small as discussed hereinafter.

As used herein, a “salt” is a pharmaceutically acceptable ionic compound composed of anions and cations so that the salt product is neutral (without charge). Sodium chloride (NaCl) is a preferred salt. Other pharmaceutically acceptable salts, by way of example and not limitation, include magnesium acetate, magnesium chloride, potassium acetate, potassium chloride, ammonium acetate, and ammonium chloride.

As used herein, “ionic strength” is the measure of total ionic concentration in solution. For purposes herein, a desired ionic strength is obtained when it prevents mercury from precipitating out of the solution for at least thirty days, and more preferably for at least sixty days, and most preferably for at least 180 days.

As used herein, “coordination compound” implies a reversible association of molecules, atoms, or ions through weak chemical bonds, and a “coordination ligand” describes an ion, ionic or molecular ensemble that forms a coordination compound with a metal ion. Ligands are generally bound to a metal ion by a coordinate bond, and are thus said to be coordinated to the ion. The nature of the coordination bond defines the reactivity of the metal complex. The crystal field theory treats all interactions in a complex as ionic and assumes that the ligands can be approximated by negative point charges. More sophisticated theories such as Ligand field theory (LFT) and Molecular orbital theory (MO) ascribe a covalent nature to the coordination bond. Coordination compounds are capable for reaction in electron transfer, ligand exchange, and associative processes. For purposes herein, a desired coordination ligand is obtained when it prevents mercury from precipitating out of the solution for at least thirty days, and more preferably for at least sixty days, and most preferably for at least 180 days.

Formulations and Stability Testing

Various aqueous solutions containing inorganic mercury (II) and a buffer were tested at multiple pH levels (from 3.0 to 7.0 with 0.5 increment steps) for stability. All solutions contained 100 mM of mercury acetate and 20 mM sodium acetate. The pH of the solution was adjusted by adding either acetic acid or sodium hydroxide. The amount of salt (NaCl) and other elements (sucrose) were varied. The solutions were sterilized, for example by filtration through a 0.22 micron filter, and stored in sterile vials. The vials were kept at room temperature and checked visually for precipitate at one week and after six months.

CHART 1
100 mM mercury acetate; 20 mM sodium acetate; 1 week stability at
room temperature:

| | pH | | | | | | | | |
|---|---|---|---|---|---|---|---|---|---|
| Formulation | 3.0 | 3.5 | 4.0 | 4.5 | 5.0 | 5.5 | 6.0 | 6.5 | 7.0 |
| a. 20 mM NaAcetate | P | P | P | P | P | P | P | P | P |
| b. 20 mM NaAcetate, | NC | P | P | P | P | P | P | P | P |
| 100 mM Sucrose | | | | | | | | | |
| c. 20 mM NaAcetate, | NC | NC | NC | NC | P | P | P | P | P |
| 300 mM Sucrose | | | | | | | | | |
| d. 20 mM NaAcetate, | P | P | P | P | P | P | P | P | P |
| 500 mM Sucrose | | | | | | | | | |
| e. 20 NaAcetate, | P | P | P | P | P | P | P | P | P |
| 75 mM NaCl | | | | | | | | | |
| f. 20 mM NaAcetate, | P | P | P | P | P | P | P | P | P |
| 150 mM NaCl | | | | | | | | | |
| g. 20 mM NaAcetate, | NC | NC | NC | NC | NC | NC | NC | NC | NC |
| 300 mM NaCl | | | | | | | | | |
| h. 20 mM NaAcetate, | NC | NC | NC | NC | NC | NC | NC | NC | NC |
| 300 mM Sucrose, | | | | | | | | | |
| 150 mM NaCl | | | | | | | | | |

NC—no changes (i.e., no visible precipitate).
P—precipitate visible

CHART 2
100 mM mercury acetate; 20 mM sodium acetate; 6 months stability at
room temperature:

| | pH | | | | | | | | |
|---|---|---|---|---|---|---|---|---|---|
| Formulation | 3.0 | 3.5 | 4.0 | 4.5 | 5.0 | 5.5 | 6.0 | 6.5 | 7.0 |
| a. 20 mM | Black | More | More | More | More + | Just | More | More | More |
| NaAcetate | dots | dots | | | red | more | red | red | red |
| | | | | | precipitate | red | precipitate | precipitate | precipitate |
| | | | | | | precip. | | | |
| b. 20 mM | White | White | Same | Same | More | Brown | Same | Same | Same as |
| NaAcetate, | precipitate | precipitate | as pH | as pH | white | precipitate | as pH | as pH | pH 6.5 |
| 100 mM | | plus | 3.5 | 4.0 | precipitate, | | 5.5 | 6.0 | |
| sucrose | | black | | | more | | | | |
| | | dots | | | black | | | | |
| | | | | | dots | | | | |
| c. 20 mM | White | Same | More | More | More | More | Black | Same | Same as |
| NaAcetate, | precipitate | as 3.5 | white | white | white | white | precipitate | as pH | pH 6.5 |
| 300 mM | | | precipitate | precipitate | precipitate | precipitate | | 6.0 | |
| sucrose | | | | | | | | | |
| d. 20 mM | Large | Larger | Larger | Larger | Larger | Larger | Larger | Larger | Larger |
| NaAcetate, | white | | | | | | | | |
| 500 mM | pellet | | | | | | | | |
| sucrose | with | | | | | | | | |
| | black | | | | | | | | |
| | dots | | | | | | | | |
| 20 mM | Small | More | More | More | More | More | More | More | Brownish |
| NaAcetate, | yellow | | | | | | | | color |
| 75 mM | precipitate | | | | | | | | |
| NaCl | | | | | | | | | |
| e. 20 mM | Very | More | More | More | More | More + | More + | More + | More + |
| NaAcetate, | little | | | | | Some | more | more | more |
| 150 mM | light | | | | | black | spots | spots | spots + |
| NaCl | yellow | | | | | spots | | | reddish |
| | precipitate | | | | | | | | color |
| | on | | | | | | | | |
| | walls | | | | | | | | |
| f. 20 mM | No visible | Same | Same | Same | Same | Same | Same | Same | Same |
| NaAcetate, | precipitate | | | | | | | | |
| 300 mM | | | | | | | | | |
| NaCl | | | | | | | | | |
| g. 20 mM | Very | Same | Same | Same | More | More | More | More | More |
| NaAcetate, | little | | | | | | | | (still |
| 300 mM | white | | | | | | | | small |
| sucrose | precipitate | | | | | | | | amount) |
| 150 mM | | | | | | | | | |
| NaCl | | | | | | | | | |

Based on the results shown in Chart 1 and Chart 2, various conclusions were drawn. First, it appears that a lower (more acidic) pH is helpful in reducing the amount of precipitate which forms. This can be seen with respect to the one week stability data where in two cases (formulas b and c which utilized sucrose and no salt), a lower pH was helpful in preventing precipitate from forming, and with respect to the six month stability data, where in just about all cases where precipitate formed, the lower the pH, the less precipitate that formed. Second, with a solution having a concentration of 100 mM of mercury acetate and 20 mM sodium acetate, the only formulation which did not precipitate at all was formulation “f” which contained 300 mM of NaCl. Thus, with a relatively high concentration of mercury acetate, more than 150 mM of NaCl salt is needed for stability. However, it is expected that at lower concentrations of mercury acetate (e.g., at 1 mM) which are more likely to be used, 150 mM of NaCl will be sufficient for stability (as discussed below). Third, the addition of sucrose appears to have at best a very minor effect in preventing the formation of precipitation.

Given the results of the stability tests shown in Charts 1 and 2, additional stability tests were conducted on various solutions where the weight volume of the inorganic mercury (II) was varied (approximately 1 mM, 2 mM, 4 mM and 12 mM), different types of inorganic mercury (mercury chloride and mercury acetate) were utilized, and different formulations (buffer plus salt, and buffer plus salt plus sucrose) were utilized. In addition, stability tests were conducted on white wine solutions taught by Vorobyeva using the same weight volumes of the mercury chloride and mercury acetate. Further, stability tests were conducted with different types of inorganic mercury (II) (mercury chloride and mercury acetate) dissolved in reagent-quality water only; i.e., without additional buffer or salt. In all cases, the solutions were kept at room temperature and checked visually for precipitate as indicated.

In the following chart (Chart 3), the wine used was a white wine (Crane Lake, Calif., Savignon Blanc, 2005). Formula 1 utilized 20 mM sodium acetate buffer, had a pH of 4.5, and included 150 mM NaCl and 300 mM sucrose. Formula 2 utilized 20 mM sodium acetate buffer, had a pH of 4.5, and included 300 mM NaCl. Formula 3 utilized reagent-quality water only.

CHART 3
All formulations stored at room temperature

| | HgCl₂- | HgAcetate- | HgCl₂ - | HgCl₂ - | HgCl2 - | HgAcetate - | HgAcetate - | HgAcetate - |
|---|---|---|---|---|---|---|---|---|
| Day | wine | wine | Formula 1 | Formula 2 | Formula 3 | Formula 1 | Formula 2 | Formula 3 |
| Day 1 | Mercury | Samples | Mercury | Mercury | Mercury | Samples | Only the | Mercury |
| | chloride | got cloudy | chloride | chloride | chloride | were | 12 mM | acetate |
| | quickly | in | quickly | quickly | quickly | cloudy | sample | quickly |
| | dissolved | proportion | dissolved | dissolved | dissolved | with | was | dissolved |
| | in | to Hg | in all | in all | in all | brown | cloudy | in all |
| | all | concentrations. | samples. | samples. | samples. | precipitate | with | samples. |
| | samples. | Brown | | | | but then | brown | |
| | | precipitate | | | | the | precipitate | |
| | | formed in | | | | precipitate | which | |
| | | all but the | | | | dissolved | quickly | |
| | | 1 mM | | | | quickly. | dissolved. | |
| | | sample. | | | | Cloudiness | | |
| | | | | | | was | | |
| | | | | | | proportional | | |
| | | | | | | to | | |
| | | | | | | Hg concentration | | |
| Day 2 | Brown | Same as | No | No | No | No | No | No |
| | precipitate | Day 1 | precipitate | precipitate | precipitate | precipitate | precipitate | precipitate |
| | formed | except | | | | | | |
| | in all | that the 12 mM | | | | | | |
| | samples. | sample | | | | | | |
| | Precipitate | had a gel- | | | | | | |
| | size | like | | | | | | |
| | proportional | consistency | | | | | | |
| | to | | | | | | | |
| | mercury | | | | | | | |
| | concentrations. | | | | | | | |
| Day 5 | No | Same as | No | No | No | No | No | No |
| | changes | Day 2 | precipitate | precipitate | precipitate | precipitate | precipitate | precipitate |
| | from | except | | | | | | |
| | Day 1 | that the | | | | | | |
| | | wine color | | | | | | |
| | | became | | | | | | |
| | | lighter | | | | | | |
| Day 7 | No | No | No | No | No | No | No | No |
| | changes | changes | precipitate | precipitate | precipitate | precipitate | precipitate | precipitate |
| | from day 5 | from day 5 | | | | | | |
| Day | No | All | No | No | No | No | No | No |
| 12 | changes | samples | precipitate | precipitate | precipitate | precipitate | precipitate | precipitate |
| | from | had gel- | | | | | | |
| | Day 7 | like | | | | | | |
| | | consistency | | | | | | |
| Day | No | No | No | No | No | No | No | Precipitate |
| 50 | changes | changes | precipitate | precipitate | precipitate | precipitate | precipitate | appeared |
| | from day | from day | | | | | | in 0.35% |
| | 12 | 12 | | | | | | sample |
| | | | | | | | | only |
| Day | No | No | No | No | No | No | No | Precipitate |
| 100 | changes | changes | precipitate | precipitate | precipitate | precipitate | precipitate | present in |
| | from day | from day | | | | | | 0.35% and |
| | 50 | 50 | | | | | | 0.117% |
| | | | | | | | | samples |
| | | | | | | | | only |
| Day | No | No | No | No | No | No | No | Precipitate |
| 180 | changes | changes | precipitate | precipitate | precipitate | precipitate | precipitate | is present |
| | from day | from day | | | | | | in all |
| | 100 | 100 | | | | | | samples. |

As a follow-up to the experiments conducted and summarized in Chart 3, a seven day stability study was conducted for 3% mercury solution in water only. The seven day stability study revealed that with the 3% mercury acetate in water (approximately 100 mM), a yellow or red precipitate formed on day 2, whereas the 3% mercury chloride remained in solution without any precipitation for the full seven days. It is believed that the mercury chloride remained in solution because it is a triatomic molecule. Since mercury chloride sublimes when it is generated during solid phase reactions, the bond between the Hg and the Cl is believed to be covalent and it remains as a triatomic molecule when mercury chloride is dissolved in water. Moreover, mercury chloride is capable of binding additional Cl ions to form tetrahedral complex HgCl₄⁻ if chloride ions are present in excess. Because HgCl₂ does not dissociate in water, its aqueous solution is stable. On the other hand, mercury acetate does dissociate in water, forming Hg and Hg(OAc) ions, as the bond between the Hg and OAc is ionic. However, non-coordinated mercury ion (Hg²⁺) is not stable in aqueous solutions and undergoes hydrolysis forming insoluble mercury oxide (yellow or red precipitate): Hg(OAc)₂+2H₂O→Hg(OH)₂+HOAc→HgO+H₂O. However, in the presence of a salt such as NaCl, one of the acetylated groups can be replaced by a chloride ion. This complex adds stability to mercury acetate in water: Hg(OAc)₂+Cl⁻=Hg(OAc)Cl+OAc⁻. Mercuric salts are used in reactions with alkenes, aromatic compounds and sulfur ligands. Reactivity of mercuric salts correlates with the electrophilicity of their mercuric ion(II), and mercury acetate is known to be a much better electrophil than mercury chloride (Lech Skulski and Piotr Wroczynski, Molecules 2001, 6, 927-958). Thus, while the mercury chloride tends to be more stable in solution than the mercury acetate (without added salt), the mercury acetate is expected to be more reactive in a biological environment than mercury chloride and hence more potent as a drug.

Based on the results contained in Chart 3, two primary conclusions were drawn. First, while mercury chloride appears generally to be more stable in solution than mercury acetate, at the relatively lower mercury levels in the solution (i.e., between 1 and 12 mM), only small amounts of salt (e.g., 150 mM NaCl) were necessary to keep the mercury acetate solution stable. Thus, there appears to be no relevant difference in the stability of the solution between the solutions using mercury acetate and solutions using mercury chloride when salt was added (thereby providing ionic strength and a coordination ligand). Second, the formulations of the invention are effective in maintaining the inorganic mercury (II) in solution, whereas the formulations of the prior art are not stable, as mercury quickly precipitates from those prior art formulations.

Compositions and Modes of Administration

In the preferred embodiment, mercury (II) compound such as mercury chloride or mercury acetate is provided in an aqueous solution at concentrations within the 1 nM to 1 M range, and more preferably within the 1 nM to 50 mM range, and even more preferably within the 0.1 mM to 10 mM range. The aqueous solution optionally includes a salt such as NaCl, and optionally also includes a buffer such as NaAcetate. Sucrose may be added, particularly for oral administration. Where added, the salt is present in a sufficient amount (typically at least 150 mM) to prevent precipitation of the mercury, and the buffer is present in sufficient amount (e.g., 10-100 mM) to maintain the pH of the solution in a desired range (e.g., between 3.0 and 7.0 and more preferably between 3.5 and 6.0, and most preferably between 4 and 5.5). Where sucrose is added, it is preferably present in a range of 100-500 mM. The aqueous solution is preferably sterilized, for example by filtration through a 0.22 micron filter, and stored in sterile vials, and has a shelf life of at least 180 days. The solution is suitable for systemic administration via injection and/or infusion, and may be administered either by bolus injection, directly through an IV catheter using a syringe, or administered by an IV pump in diluted form continuously during the day. Formulations for injection may be presented in unit dosage form, e.g., in ampoules or in multi-dose containers, with an added preservative.

Treatment and Dosing Schedules.

A single treatment course of inorganic mercury (II) in the acute or chronic management of cancer might consist of either its single administration or repeated administration preferably for 1-365 days. Each treatment course might be repeated a number of times with generally 2-90 days (and more preferably 5 to 30 days) of intervals in between. The duration of a single treatment course and the required number of such treatment courses will vary and be determined by a cancer type, stage of the disease, bulk amount of tumor masses present at initiation time and individual patient's response. The magnitude of a therapeutic dose of inorganic mercury (II) will also vary with the severity of the condition to be treated and the route of administration. Doses will be derived from known toxicity information that will include but not limited to bioavailability data, minimal risk levels (MRL), no-observed-adverse events levels (NOAEL), less serious and serious lowest-observed-adverse-events levels (LOAEL). The therapeutic doses for short-term and long-term treatments will generally be between MRL and serious LOAEL. The doses are calculated in the micromole Hg²⁺/kg/day units. Common conversion formulas will be used for calculations of daily doses for different species including a human, dog or cat (Cancer Chemotherapy Reports 50(4): 219 (1966)). The daily dose might be administered continuously via IV, or through repeated increments during a day, or once a day, or a doubled daily dose every other day. Generally, the daily dose for inorganic mercury (II) will range from about 0.001 to 100 micromole Hg²⁺kg/day for humans and corresponding mg/m² dose equivalences for other species.

Here is an exemplary course of treatment of a human patient with leukemia, lymphoma, or solid cancer by administration via injection into the bloodstream of a composition comprising mercury (II) inorganic compound in an aqueous solution. Human MRL for intermediate oral exposure to mercuric chloride derived from 26 weeks study in rats is 0.002 mg/kg/day or approximately 0.007 micromole Hg²⁺/kg/day, NOAEL is 0.027 mg/kg/day or approximately 0.09 micromole Hg²⁺kg/day, and the acute exposure LOAEL for serious effects in humans is known to be 30 mg/kg/day or approximately 100 micromole Hg²⁺/kg/day (Toxicological profile of Mercury, March 1999, CAS#7439-97-6, Agency for Toxic Substances and Disease Registry). IV administration should be considered to be at least as effective as oral administration, and thus, similar or smaller IV dosages may be used. Since inorganic mercury is preferentially absorbed by cancer cells (Omura et al., 1996, Acupunct. Electrother Res; 21(2):133-60, Kanai et al., 1980, Shinshu Igaku Zasshi; 28(2):221-228), its toxicity effects in cancer patients will be observed at higher mercury levels than the levels expected for healthy individuals. Thus, the therapeutic dose range for 180-day treatment course of human cancer with oral administration will likely be between 0.1-10 micromoles Hg²⁺/kg/day. Again, since IV administration is expected to be at least as effective as oral administration, similar or smaller dosages may be used (e.g., between 0.01 and 10 micromoles Hg²⁺/kg/day.) Higher doses might be used for a shorter than 180-day treatment period. Lower doses might be used for a longer than 180-day treatment period. The therapeutic dose is given by injection into the bloodstream once a day. The course of treatment may continue until remission is observed, or when side effects should become serious, or when it reaches 365 days. It is preferred that the duration of the first treatment course be 30-180 days depending on the severity of condition. If after 180 days of treatment, a patient does not respond favorably, the treatment may be stopped. The course of treatment may be repeated multiple times with about 2-90 day intervals (and more preferably 5 to 30 days) between courses.

The method of the present invention may be used to treat primary cancers as well as metastatic tumors. The primary cancer may be a solid tumor or disperse cancer. The solid tumor may include but is not limited to lung cancer, breast cancer, ovarian cancer, prostate cancer, bowel cancer, brain cancer, testicular cancer, colon cancer, liver cancer, kidney cancer, pancreas cancer, skin cancer, neck cancer, uterus cancer, bone cancer. The disperse cancer may be leukemia or lymphoma. Whenever possible, it is preferred that the bulk of tumor be removed surgically prior to treatment to optimize its outcome.

In a specific embodiment, the composition of the present invention is administered systemically to the mammal (e.g., human, dog, cat) at least once. In a more specific embodiment, the method comprises an initial administration of the composition of the present invention and at least one subsequent administration of said composition generally 2-90 days (and more preferably 5 to 30 days) after initial administration. In yet another specific embodiment, the method further comprises subsequent administration of said composition at least two times after initial administration.

In a particular embodiment, more than one mercury (II) compound may be administered. The compounds may be administered sequentially or simultaneously.

While systemic administration of an effective systemic dosage of inorganic mercury (II) via injection or transfusion is preferred, any suitable route of administration may be employed for providing the patient with an effective systemic dosage of inorganic mercury (II) (See, Remington's Pharmaceutical Sciences, J. P. Remington, Easton, Pa.: Mack Pub. Co., 18^{th} th Edition, 1990).

### Examples

As described in the previously incorporated parent application U.S. Ser. No. 11/324,850, a formulation containing 1 mM mercury acetate, 20 mM sodium acetate buffer, pH 4.5, with 150 mM NaCl salt, and 100 mM sucrose was given orally to four subject dogs in a dose of 0.5 micromole Hg²⁺/kg/day for various treatment periods, and the results indicated effectiveness of the formulation. It is believed that systemic administration via injection or infusion can be used to reduce the dosage relative to an oral administration.

There have been described and illustrated herein several embodiments of stable aqueous compositions containing inorganic mercury (II) and methods of using the same. While particular embodiments of the invention have been described, it is not intended that the invention be limited thereto, as it is intended that the invention be as broad in scope as the art will allow and that the specification be read likewise. Thus, while particular inorganic mercury (II) compounds have been disclosed, it will be appreciated that others may be used as well. In addition, while particular buffers and salts have been disclosed, it will be understood other buffers and salts can be used. It will therefore be appreciated by those skilled in the art that yet other modifications could be made to the provided invention without deviating from its spirit and scope as claimed.

What is claimed is:

1. A liquid composition suitable for injection into a mammal in need thereof, comprising an aqueous solution of:
an inorganic mercury (II) containing compound present in an amount between 0.1 nanomole and 50 millimoles Hg²⁺;
a buffer which stabilizes the pH of the liquid composition at a desired pH between 3.0 and 7.0; and
at least one salt provided in amounts which provide sufficient ionic strength to said solution to prevent precipitation from said solution for at least thirty days of a visible precipitate containing at least some of said mercury.

2. A liquid composition according to claim 1, wherein:
said inorganic mercury (II) containing compound is chosen from mercury acetate and mercury chloride.

3. A liquid composition according to claim 2, wherein:
said solution has a pH of between 3.5 and 6.0.

4. A liquid composition according to claim 3, wherein:
said solution is suitable for injection into the bloodstream of the mammal.

5. A liquid composition according to claim 3, wherein:
said solution is suitable for parenteral injection into the mammal.

6. A liquid composition according to claim 2, wherein:
said buffer is NaAcetate.

7. A liquid composition according to claim 6, wherein:
said NaAcetate is present in an amount of between 10 and 30 mM.

8. A liquid composition according to claim 7, wherein:
said salt is present in an amount of more than 150 mM.

9. A liquid composition according to claim 8, wherein:
said salt is NaCl and is present in an amount of at least 300 mM.

10. A dose of said liquid composition according to claim 1, wherein:
said dose is for a human having a weight, and said dose contains between 0.001-100 micromole Hg²⁺/kg of the human.

11. A method comprising injection a dose of said liquid composition according to claim 10 into the bloodstream of the human.

12. A liquid composition suitable for injection into a human, comprising an aqueous solution of:
an inorganic mercury (II) containing compound present in an amount between 0.1 nanomole and 50 millimoles Hg² and chosen from mercury acetate and mercury chloride;
a NaAcetate buffer which stabilizes the pH of the liquid composition at a desired pH between 4.0 and 5.5; and
at least one salt provided in amounts which provide sufficient ionic strength to said solution to prevent precipitation from said solution for at least thirty days of a visible precipitate containing at least some of said mercury, said amounts being greater than at least 150 mM,
said solution being suitable for parenteral injection or injection into the bloodstream of the human at a dose containing between 0.001-100 micromole Hg²⁺/kg of the human per day.

13. A method comprising injecting a dose of said liquid composition according to claim 12 into the bloodstream of the human.

14. A liquid solution suitable for injection into a human, comprising:
an inorganic mercury (II) containing compound present in an amount between 0.1 nanomole and 50 millimoles Hg²⁺; and
water,
wherein said solution is sufficiently stable such that there is no precipitation from said solution for at least thirty days of a visable precipitate containing at least some of said mercury.

15. A liquid solution according to claim 14, further comprising:
said inorganic mercury (II) containing compound is mercury acetate, and said liquid solution further comprises at least one salt provided in amounts which provide sufficient ionic strength to said solution to prevent precipitation from said solution for at least thirty days of a visible precipitate containing at least some of said mercury.

16. A liquid solution according to claim 15, wherein:
said solution has a pH of between 3.5 and 6.0, and said salt is present in an amount of more than 150 mM.

17. A liquid solution according to claim 14, wherein:
said solution is suitable for injection into the bloodstream of the mammal or for parenteral injection into the mammal.

18. A dose of said liquid solution according to claim 14, wherein:
said dose is for a human having a weight, and said dose contains between 0.001-100 micromole Hg²⁺/kg of the human.

19. A method comprising injection a dose of said liquid solution according to claim 18 into the bloodstream of a human.

20. A liquid composition suitable for injection into a mammal in need thereof, comprising an aqueous solution consisting essentially of:
an inorganic mercury (II) containing compound present in an amount between 0.1 nanomole and 50 millimoles Hg²⁺;
a buffer which stabilizes the pH of the liquid composition at a desired pH between 3.0 and 7.0; and
at least one salt provided in amounts which provide sufficient ionic strength to said solution to prevent precipitation from said solution for at least thirty days of a visible precipitate containing at least some of said mercury.
